(19) United States
(12) Patent Application Publication
(10) Pub. No.: US 2022/0009899 A1
(43) Pub. Date: Jan. 13, 2022

(54) PROCESS AND COMPOUNDS FOR PREPARATION OF CANNABINOIDS

(71) Applicant: EMBIO LIMITED, Mumbai (IN)

(72) Inventors: Gollapudy SUBRAHMANYAM, Hyderabad (IN); Sunil JOSHI, Mahad (IN); Nilesh Liladhar BONDE, Pune (IN); Jinesh Gajanand BHATT, Dungarpur (IN); Makarand Sitaram GORE, Ahmadnagar (IN)

(73) Assignee: EMBIO LIMITED, Mumbai (IN)

(21) Appl. No.: 17/294,095

(22) PCT Filed: Feb. 20, 2019
(86) PCT No.: PCT/IB2019/051371

§ 371 Date: May 14, 2021

(30) Foreign Application Priority Data
Nov. 14, 2018 (IN) 201821042737

(51) Int. Cl. C07D 311/80; C07C 311/53; C07C 29/16

(52) U.S. Cl. CPC C07D 311/80; C07C 2601/16; C07C 29/16; C07C 311/53

(57) ABSTRACT

The invention involves condensation of various derivatives of cyclic alkene alcohols of Formula II or Formula III where, R¹ is alkyl, aryl, alkyl aryl or heterocyclic carbamoyl. R² is either R¹ as mentioned earlier or an acyl group, —C(═O)—R³ where, R³ is selected from a group comprising (subst)C₁-C₁₂ alkyl, (subst)aryl, alkyl aryl with olivetol to get (−)-trans-Δ⁹-tetrahydrocannabinol (also known as Dronabinol, Formula I). The process disclosed provides high purity of dronabinol at crude stage making it easy for purification.

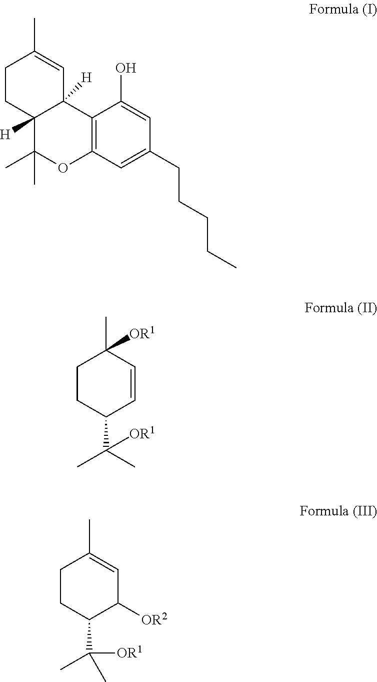

### TECHNICAL FIELD OF THE INVENTION

The present invention relates to process for preparation of Cannabinoids. The invention particularly describes the process for preparation of (−)-trans-Δ⁹-tetrahydrocannabinol from novel precursors.

### BACKGROUND OF THE INVENTION

The principal psychoactive component of cannabis plant is (−)-trans-Δ⁹-tetrahydrocannabinol. It is isolated from cannabis plant and is also synthesized by numerous research groups.

JACS 62, 2402 (1940) reported acid catalyzed dehydration of cannabidiol to tetrahydrocannabinol by using either Lewis acids or mineral acids. The said process requires heating the reaction mass at very high temperature and yielding complex mixture of compounds and is a laborious exercise from commercial point of view.

Helv. Chim Acta 52, 1102 (1969) reported synthesis of (−)-trans-Δ⁹-tetrahydrocannabinol, from cannabidiol which was prepared using olivetol and menthe-2,8-diene-1-ol using DMF: dineopentyl actal. The reported invention does not give pure cannabidiol and contains other isomeric compound making the isolation of cannabidiol difficult. It is also silent with respect to the product purity and related impurities.

There was further work with different cyclic olefins as citral and verbinol by Mechoulam et al in JACS 94, 6159 (1972). With citral reaction the product gave mixture of cis-Δ⁹-tetrahydrocannabinol along with desired product. With verbinol the product gave desired product but the SOR mentioned was −245° (CHCl₃) with 44% yield.

Although there is lot of literature for preparation of tetrahydrocannabinol by total synthesis or new methodology reported by many research workers, a few synthetic works are cited herein as, Herchel Smith et al JACS 89, 4551 (1967), Fahrenholtz et al JACS 88, 2079 (1966), D. A. Evans et al JACS 121, 7582 (1999), D. H. Dethe et al Chem Comm 51, 2871 2015 and U.S. Pat. No. 7,323,576. None of the said process has commercial viability and requires multiple purifications by column chromatography and to add more to this the atom economy of the conversion is very poor.

From the literature it is evident that several methods have commonly used either mentha-2,8-diene-1-ol or menth-2-ene-1,8-diol as cyclic olefins.

The U.S. Pat. No. 5,227,537 reported the use of menth-2-ene-1,8-diol as cyclic olefin and condensation of it with olivetol using pTSA. The said invention reported the isolation of open chain condensed product containing 3-hydroxy group with 46% yield. The yielded compound was further dehydrated using zinc chloride which gave Δ⁹-tetrahydrocannabinol. The process involves purification by column chromatography at both stages and is silent about yield and related impurities.

Further to this, protecting menth-2-ene-1,8-diol by making its acyl derivative and condensing the isolated diester with olivetol was described in U.S. Pat. No. 7,186,850 B2.

The U.S. Pat. No. 7,323,576 B2 reported synthesis of cis-(1S,6R)-6-(2-hydroxyprop-2-yl)-3-methylcyclohex-2-en-1-ol, here after menth-1-ene-3,8-diol. The said patent further reports synthesis of tetrahydrocannabinol using the above-mentioned cyclohexene. The process disclosed is silent with respect to purity of the isolated compound and report moderately low yield.

The United States Patent Application number US2017/0008869A1 discloses condensation of 4,6-dihaloolivetol with menth-2,8-diene-1-ol as to get dihalo derivative of cannabidiol, which upon dehalogenation and cyclization gives tetrahydrocannabinol. Even though the synthetic pathway was elaborated, the said application does not disclose quality of the product obtained.

The prior arts detailed above demonstrate the difficulties of manufacturing (−)-trans-Δ⁹-tetrahydrocannabinol, in high yield, high Stereo-specificity, or both. The causes of these difficulties can include the non-crystalline nature of the materials which renders them difficult or impossible to separate and purify without chromatography. Also, the aromatic portion of the (−)-trans-Δ⁹-tetrahydrocannabinol molecule is sensitive to oxidation and has concern with respect to thermodynamic stability.

Therefore, there is a need for a process for the synthesis of (−)-trans-Δ⁹-tetrahydrocannabinol which can give high yield of the product along with high stereo-specificity and reduces the unwanted isomeric impurities.

### SUMMARY OF THE INVENTION

The present disclosure relates to the process for preparation of (−)-trans-Δ⁹-tetrahydrocannabinol, with very high purity and with almost negligible formation of related impurities like Δ⁸-tetrahydrocannabinol and cannabidiol. The disclosure further relates the process of synthesizing the desired compound without losing the atom economy, thus making it commercially viable.

The present invention relates to a new methodology for synthesis of (−)-trans-Δ⁹-tetrahydrocannabinol, by acid catalyzed cyclization of olivetol with differently substituted cyclohexene derivatives. The desired product is obtained in high yield and purity at crude stage, the major advantage being that the said process eliminates Δ⁸-tetrahydrocannabinol formation.

In one embodiment, the invention is a process for protection of menth-2-ene-1,8-diol Formula VII with n-alkyl, branched alkyl, substituted or un-substituted aryl, alkyl aryl, heteroaryl derivative of sulfonyl isocyanate in organic solvents. The reaction is carried out in presence or absence of organic and/or inorganic base, to derive following general structure of Formula IV, wherein R¹ is n-alkyl, branched alkyl, substituted aryl, alkyl aryl or heterocyclic derivative of sulfonyl isocyanate.

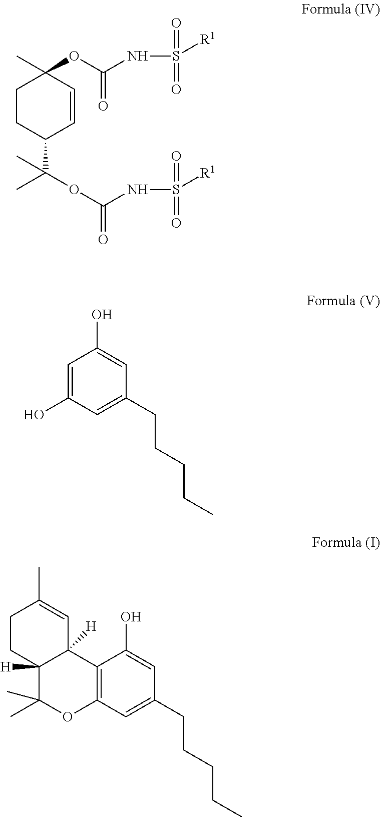

Condensation of compound of Formula IV with olivetol Formula V in the presence of mineral, organic acid or Lewis acid or mixture thereof gives desired isomer of tetrahydrocannabinol Formula I.

In another embodiment, the invention is the process for conversion of menth-1-ene-3,8-diol Formula VI to its acyl derivatives Formula III by condensation with either carboxylic acids or respective acid chloride. Obtained acyl derivatives are condensed with olivetol in the presence of organic, mineral or Lewis acid in aprotic organic solvent to give (−)-trans-Δ⁹-tetrahydrocannabinol of [Formula I], wherein R² and R³ are any alkyl, substituted or un-substituted aryl or heterocyclic derivatives.

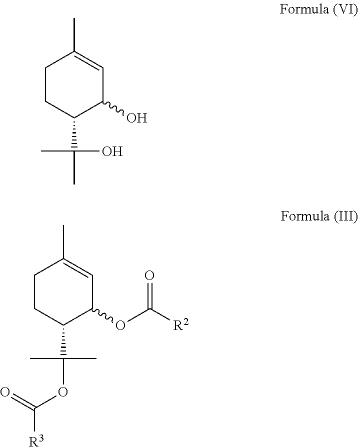

Yet another embodiment of the present disclosure relates to the process for preparation of menth-1-ene-3,8-diol Formula VI from menth-2-ene-1,8-diol Formula VII, followed by conversion of said compound of Formula VI to its diesters of Formula III as per the above revealed process. The compound of Formula III obtained was converted to (−)-trans-Δ⁹-tetrahydrocannabinol as per above disclosed invention.

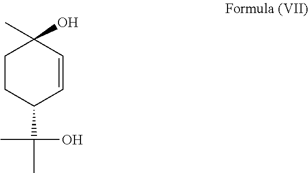

In one more embodiment, the diol of Formula VI is activated by reaction with alkyl, aryl, alkyl aryl or heteroaryl sulfonyl isocyanates (as per the first embodiment) and obtained compound is either isolated Formula VIII or used in situ for condensation with olivetol of Formula V to give (−)-trans-Δ⁹-tetrahydrocannabinol of Formula I, wherein R¹ is selected from n-alkyl, branched alkyl, aryl, alkyl aryl, heterocycle.

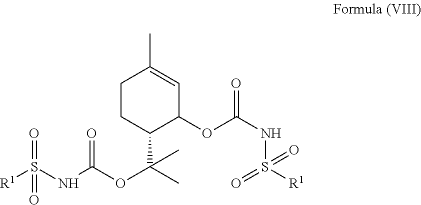

Processes of present disclosure provide number of advantages over current methods disclosed in the prior art as listed below. During conversion to Δ⁹-tetrahydrocannabinol, isomeric Δ⁸-tetrahydrocannabinol was almost absent due to very short reaction time for cyclization, thereby eliminating risk for the isomerization. The hydroxyl group at 8^{th} position of either menth-2-ene-1,8-diol or menth-1-ene-3,8-diol was protected/activated either by acyl or carbamoyl group, accelerating the rate for cyclization reaction, resulting in near absence of open chain compound (cannabidiol) even though less than equimolar quantity of Lewis acid is used. Yields in every disclosed process were almost 90-100% at crude stage. The overall yield by the process for pure (−)-trans-Δ⁹-tetrahydrocannabinol was 72%-80%.

### BRIEF DESCRIPTION OF THE DRAWINGS

The foregoing and other features of embodiments will become more apparent from the following detailed description of embodiments when read in conjunction with the accompanying drawings.

FIG. 1 represents the process for preparation of (−)-trans-Δ⁹-Tetrahydrocannbinol from sulfonyl carbamates.

FIG. 2 is a process for preparation of compound of Formula III from compound of Formula VI.

FIG. 3 represents Preparation of phenyl acetyl diester followed by (−)-trans-Δ⁹-tetrahydrocannabinol.

FIG. 4 is a schematic representation of preparation of phenyl acetyl diester followed by tetrahydrocannabinol.

FIG. 5 represents the preparation of (−)-Trans-Δ⁹-Tetrahydrocannabinol from 3,8-carbamate olefin.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

The present disclosures relate to process for preparation of tetrahydrocannabinol, with purity as per pharmacopeia without sacrificing the atom economy. Further to this, the disclosed procedure successfully eliminates the risk for close eluting impurities, as presence of such impurities is relatively low compared to above mentioned prior art processes.

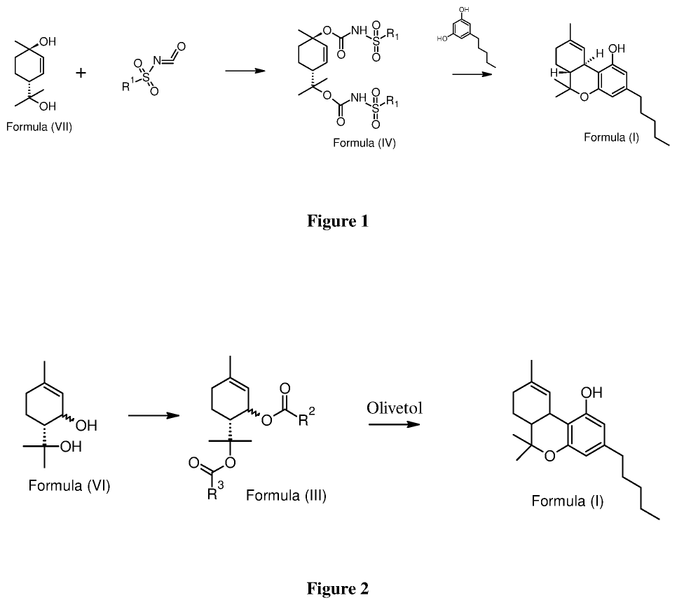

In one embodiment, the present disclosure relates to a process for the preparation of (−)-trans-Δ⁹-tetrahydrocannabinol from compound of Formula IV. FIG. 1 represents the process for preparation of (−)-trans-Δ⁹-Tetrahydrocannbinol from sulfonyl carbamates. The said compound of Formula IV is synthesized by reacting menth-2-ene-1,8-diol Formula VII with alkyl/aryl/alkyl aryl/heteroaryl sulfonyl isocyanate (R¹—S(O₂)—N═C═O), as illustrated in FIG. 1, wherein R¹ is alkyl/aryl/alkyl aryl/heteroaryl group. Alkyl groups can be normal chain, branched chain optionally carrying one or more substituents. Aryl group can be phenyl group or a naphthyl group, optionally carrying one or more nuclear substituents independently selected from halo, cyano, sulfonate, carboxylate, carboxylic acid, aldehyde, keto, nitro, tertiary amino, trichloromethyl and trifluoromethyl. Alkyl aryl can be benzyl, substituted benzyl on alkyl or aryl system with either enforcing inductive or resonance stability/instability to the reagent. The heteroaryl group can be fused ring or single ring with one or more hetero atoms which can be sulfur, nitrogen, oxygen with/without one or more substituents. The heteroaryl ring can be attached through carbon or hetero atom to the sulfur atom of sulfonyl isocyanate. Largely on grounds of cost and efficiency, derivatives of phenyl sulfonyl isocyanates are preferred and amongst them, p-toluene sulfonyl isocyanate is most preferred, i.e. R¹ being p-toluene.

Reaction of sulfonyl isocyanates with compound of Formula VII is carried out in an organic solvent which is one of tetrahydrofuran, dioxane, toluene, halogenated solvent or mixture thereof preferably halogenated solvents and more preferably methylene chloride.

Conversion of compound of Formula VII to Formula IV, is carried out at −25 to +75° C., preferably at −25° to 30° C. and more preferably at 0 to 20° C.

After the conversion the compound of Formula IV is either isolated or used in situ for condensation with olivetol Formula V as illustrated in FIG. 1 to give tetrahydrocannabinol of Formula I. The reaction for conversion is carried out using equimolar quantities of both the compound of Formula IV and V, in halogenated solvent preferably methylene chloride at temperature from −25° C. to 25° C., preferably at −10° to 0° C. The reaction is carried out using any aromatic acid, mineral acids or Lewis acids or mixture thereof, preferably Lewis acids. Lewis acid is one of ZnCl₂, Ti (IV) isopropoxide, tri isobutyl aluminium, or metal triflates. Metal in metal triflates can be Zinc, Ytterbium, Yttrium, and Scandium. Boron trifluoride as ether, acetic acid, acetonitrile complex is a preferred Lewis acid and boron trifluoride etherate is the more preferred one. The said condensation can be carried out optionally in presence of dehydrating agents. Dehydrating agents are sulfate salts of alkali or alkaline earth metals the more preferred is Magnesium Sulfate.

An embodiment of the invention is the process for preparation of (−)-trans-Δ⁹-tetrahydrocannabinol from compound of Formula III, wherein compound of Formula III, is prepared from compound of Formula VI. The chemical conversion is disclosed in FIG. 2

FIG. 2 is a process for preparation of compound of Formula III from compound of Formula VI. According to FIG. 2, compound of Formula VI can be prepared by the procedure disclosed in Tetrahedron 43 5537 (1987). The said hydroxyl group at third and eighth positions was converted to its acyl derivative. Here, in allylic and tertiary hydroxyl functionalities of compound of Formula VI is optionally differently acylated. Acylation can be carried out by condensation of compound of Formula VI with either carboxylic acids of aliphatic, aromatic, alkyl aryl, heterocyclic compound preferably alkyl aryl and more preferably benzyl, dibenzyl group. Condensation of compound of Formula VI can be carried out by using carboxylic acids or its respective acid chloride.

Condensation of carboxylic acids is carried out in organic solvents and coupling agents can be carbodiimides, Phosphonium based coupling agent (e.g. APO, PyAOP, BrOP, PyClop, FDPP, DEPBT, BDP), Uronium (e.g. BCC, TDBTU, TNU, TPTU, TSTU, HAPyu, TAPipU, CIP, HATU, TBTU) or imminium based (BOM, BDMP) coupling agent and in the presence of organic bases which includes dimethyl amino pyridine and hydroxy benzotriazole. The said acylation can be carried out in inert organic solvents which include tetrahydrofuran, methyl tetrahydrofuran, dioxan or halogenated solvents (methylene chloride, ethylene chloride, chloroform) preferably in tetrahydrofuran.

Condensation of carboxylic acid is carried out by synthesizing the corresponding acid chloride. Synthesis of acid chloride is carried out using halogenating agents as thionyl chloride, oxalyl chloride, phosphorous pentachloride, phosphorous trichloride or phosphorous oxychloride preferably thionyl chloride and optionally in organic solvent. Organic solvent is one of chlorobenzene, toluene, xylene, nitrobenzene, chloroform, methylene chloride or ethylene chloride. The preferable solvent for preparing un-substituted or substituted alkyl aryl carboxylic acid of Formula IX is toluene due to commercial feasibility from cost and yield perspective. R₄ in Formula XI is H, aromatic, aliphatic, heteroaryl substituent, optionally with additional substituents being Cl, Br, I or NO₂ groups. R₅ is one among H, Cl, Br, I, NO₂, alkyl, aryl, heteroaryl substituents.

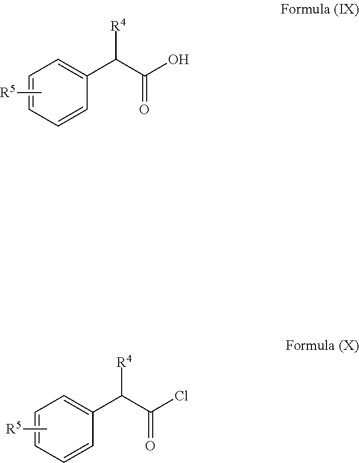

Condensation of acid chloride of Formula X with diol of Formula VI can be carried out in the presence of organic bases or weak inorganic bases optionally in organic solvents to get diesters of Formula III and preferably diester of Formula XI.

Organic bases used is one of pyridine, methyl pyridine, pyrrolidine, trimethyl amine, triethyl amine, tripropyl amine, diisopropyl ethyl amine, N-methyl morpholine, triethyl amine, DAMEDA, TAMEDA, DABCO or a combination thereof. Organic solvents are Methylene chloride, ethylene chloride, toluene, or organic bases preferably in pyridine, methyl pyridine and pyrrolidine more preferably pyridine. The reaction is carried out optionally in the presence of more nucleophilic base preferably dimethylaminopyridine. Temperature of the reaction can be maintaining from 0° C. to reflux temperature preferably between 25° C.-35° C.

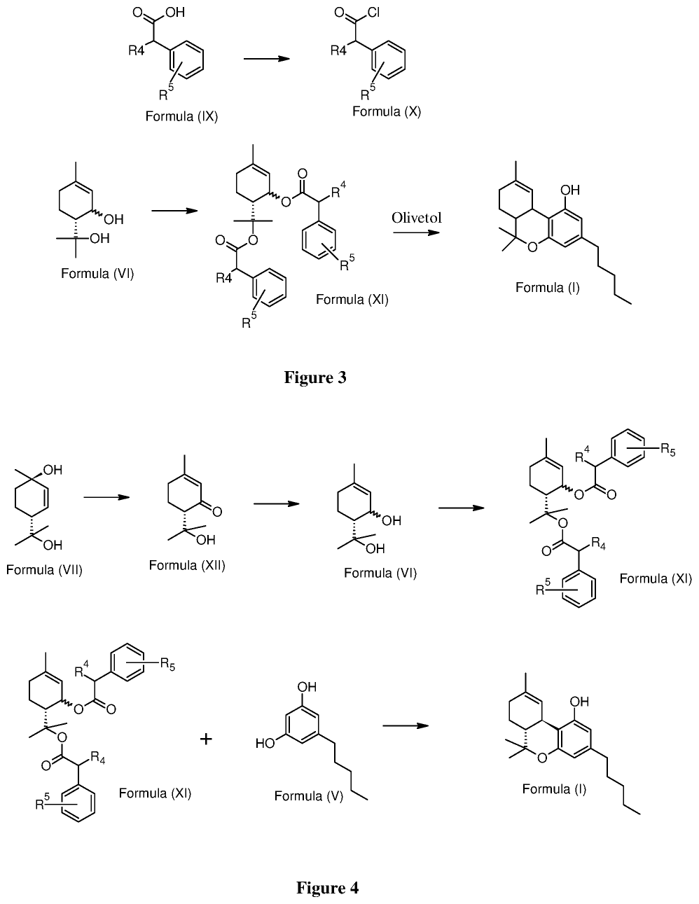

Diester compound of Formula XI is condensed with olivetol of Formula V in organic solvent in presence of Lewis acids and in presence or absence of dehydrating agent to get (−)-trans-Δ⁹-tetrahydrocannabinol as represented in FIG. 3.

Boron trifluoride etherate is the preferred Lewis acid used. Use of Boron trifluoride can be from 20 mole % to 200 mole % preferably 50 to 100 mole %. The reaction temperature for condensation and dehydration is from −50° to 50° C. preferably −10° to 0° C. After completion of reaction, the reaction mass is washed with aqueous alkali metal carbonate solution or alkali metal hydroxides preferably alkali metal hydroxide. The alkali metal hydroxide is one of lithium, sodium or potassium hydroxides more preferably sodium hydroxide.

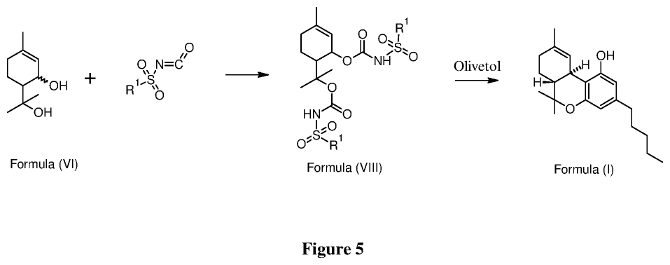

In yet another embodiment, the present disclosure relates the new method for synthesis of menth-1-ene-3,8-diol of Formula VI from menth-2-ene-1,8-diol of Formula VII as depicted in FIG. 4.

Compound of Formula VII is treated with oxidizing agent there by conversion to compound of Formula XI. The oxidizing agent is chromium (VI) based or Mn (IV) or Mn (VII) based oxidizing agents. It is one of sodium chromate, potassium chromate, Pyridinium dichromate, pyridinium chlorochromate, potassium permanganate, manganese dioxide. The preferred oxidizing agent is pyridinium chlorochromate. Solvent for oxidation is organic aprotic polar/non-polar solvents like methylenechloride, benzene, ethylene chloride, chloroform preferably methylene chloride. Reaction is carried out between −50° C. to +50° C. preferably between 0° C. to 30° C. and more preferably between 10° C. to 20° C.

Compound of Formula IV is synthesized from compound of Formula XII using hydride based reducing agents. The reducing agent is a borohydride such as NaBH₄, LiBH₄, KBH₄, NaBH₃CN, BH₃.THF or aluminium hydrides such as LiAlH₄, NaAlH₂(OC₂H₅OCH₃)₂ preferably sodium borohydride as they are safe to handle on commercial scale. The said reduction is carried out using Luche reduction condition i.e. in presence of ceric (III) chloride, Lanthanide (III) chloride, scandium triflate preferably ceric (III) chloride. The solvent for reduction is one of methanol, ethanol, 2-propanol or tetrahydrofuran, dioxane preferably methanol and 2-propanol and more preferably methanol which is economically more feasible.

Diester compound of Formula XI and (−)-trans-Δ⁹-tetrahydrocannabinol of Formula I is prepared by the earlier disclosed embodiments.

In yet another embodiment, the present disclosure relates to a process for the preparation of (−)-trans-Δ⁹-tetrahydrocannabinol from compound of Formula VIII. The said compound of Formula VIII was synthesized by reacting menth-1-ene-3,8-diol of Formula VI with alkyl/aryl/heteroaryl sulfonyl isocyanate (R¹—SO₂—N═C═O), as illustrated in FIG. 5, wherein, R¹ in sulfonyl isocyanate which is alkyl/aryl/alkyl aryl/heterocyclic compound. The alkyl group is n-alkyl or branched alkyl with or without functional group/s enhancing overall reactivity of alkyl sulfonyl isocyanate. The aryl group is with/without substituents resulting either in activation or deactivation of overall reactivity for derived isocyanates. The heterocyclic ring can be saturated or unsaturated and is either fused ring or single ring. In the fused ring, the system connected is either of another heterocyclic ring or homocyclic ring. The said heterocyclic ring is one having three members to ten members with one or more heteroatoms. Herein heteroatoms can be sulfur, nitrogen, oxygen. Additionally, the said heterocyclic ring can be connected to sulfur of sulfonyl isocyanate through carbon or heteroatom of heterocyclic ring. The heterocyclic ring is connected directly or through another aliphatic spacer to sulfonyl isocyanate thereby inducing the reactivity to sulfonyl isocyanate functionality. Considering the cost, ease of handling, commercial availability and environmental chemical impact substituted or un-substituted aryl sulfonyl isocyanates are considered for screening.

Reaction of aryl sulfonyl isocyanate with compound of Formula VI is carried out in aprotic solvents that include tetrahydrofuran, dioxane, toluene, xylene, chloroform, methylene chloride, dimethyl formamide, cyclohexane, hexane, heptane and ether. The reaction is carried out in the absence/presence of base which is either an organic or an inorganic base. The aryl sulfonyl isocyanate reacted preferably in the absence of base in halogenated solvent preferably methylene chloride. Obtained carbamoyl compounds are optionally isolated and used for next stage or preferably used for in situ condensation.

The compound of Formula VIII, is used in situ for condensation with olivetol in aprotic solvent to give (−)-trans-Δ⁹-Tetrahydrocannabinol. The said solvent is either halogenated or ethereal, preferably halogenated solvent and more preferably methylene chloride. Reaction is carried out at −50° C. to 30° C., preferably at −10° C. to 10° C. and more preferably at −10° C. to 0° C. The reaction is carried out in the presence of Bronsted or Lewis acid, preferably Lewis acid more preferably boron trifluoride as its etherate. The obtained (−)-trans-Δ⁹-Tetrahydrocannabinol is purified by column chromatography.

The following examples are offered to illustrate various aspects of the present invention and are not intended to limit or define the present invention in any matter.

### Example 1: Process for Preparation of (−)-Trans-Δ⁹-Tetrahydrocannabinol from Menth-2-Ene-1, 8-Diol, Through its p-Toluene Sulfonyl Carbamate

### Part A: Preparation of menth-2-ene-1,8-bis[(4-methyl phenyl)sulfonylcarbamate]

A 100 mL round bottom flask provided with magnetic stirrer bar was oven dried, fitted with an addition funnel and was cooled under stream of Nitrogen. Menthylene chloride (60 mL) and Menth-2-ene-1,8-diol (11.75 mmol, 2 g) was charged to get homogeneous solution. p-Toluene sulfonyl isocyanate (29.3 mmol, 5.8 g) was added at 0-5° C. Progress of the reaction was observed by TLC. After completion of reaction, aqueous ammonium chloride solution (20 mL) was added. The organic layer was dried over sodium sulfate and used as such for the next step.

### Part B: Preparation of (−)-trans-Δ⁹-tetrahydrocannabinol

Organic layer from part-A was added to oven dried 250 mL round bottom flask provided with magnetic stirrer bar with addition funnel. Olivetol (11.75 mmol, 2.1 g) was added to the flask and the contents were cooled to −10° C. to −5° C. BF₃.OEt₂ (11.75 mmol, 3.5 g) was added in 5 minutes. After completion of reaction, the reaction mass was quenched with aqueous 2% Sodium hydroxide. Methylene chloride layer was separated and dried over sodium sulfate. The obtained organic layer was concentrated under vacuum to get syrupy oil. Yield: 90%, HPLC: 74% (−)-trans-Δ⁹-tetrahydrocannabinol.

Part C: Purification: Crude compound obtained in Part B was purified by column chromatography. The solvent used for elution was 1% DIPE-pet ether to 10% DIPE-pet ether. Yield: 60%, HPLC: 96%.

### Example 2: Process for Preparation of Menth-1-ene-3,8-diol from menth-2,8-diene-1-ol

### Part A: Preparation of isopiperitenone [(6S)-6-isopropenyl-3-methylcyclohex-2-en-1-one]

A 250 mL Round bottom flask provided with overhead stirrer was charged with menth-2,8-diene-1-ol (32.8 mmol, 5 g) and methylene chloride (75 mL). Pyridinium chlorochromate (44.5 mmol, 9.6 g) was added at 0° C.-10° C. in lots. The mixture was stirred for 30 minutes and concentrated under reduced pressure to get brownish semi solid. Water (100 ml) was added and extracted with isopropyl ether (3×100 mL). The organic layer was separated, dried over sodium sulfate and concentrated under vacuum to give oily compound. Yield 91%; GC 93.2%; IR: 1668, 2936 cm⁻¹. NMR δ 5.816 (s, 1H), 4.865 (s, 1H), 4.681 (s, 1H), 2.859-2.899 (dd, 1H), 2.347-2.211 (m, 2H), 2.079-1.997 (m, 1H), 1.983-1.906 (m, 1H), 1.887 (s, 3H), 1.671 (s, 3H).

### Part B: Preparation of (6s)-3-methyl-6-(2-methyloxiran-2-yl)cyclohex-2-en-1-one

A 250 mL Round bottom flask with magnetic stirrer bar and pressure equalizing addition funnel was charged with isopiperitenone (33.3 mmol, 5 g) and chloroform (40 ml). Meanwhile mCPBA (50 mmol, 11 g, assay 75%) was dissolved in chloroform (110 mL). The resulting mCPBA solution was added through addition funnel in 90 minutes. Reaction mass was quenched with aqueous 10% sodium bicarbonate. Organic layer was separated and washed with aqueous 2% sodium metabisulfite, followed by concentration under reduced pressure to give colourless oil, which is further purified by column chromatography. Yield: 96% GC 96.54%, IR 3040, 2975, 2932, 1671, 1436, 1379 cm⁻¹, NMR δ 5.780 (s, 1H), 2.691 (d, J=4.8 Hz, 1H), 2.636 (d, J=5 Hz, 1H), 2.394 (m, 1H), 2.338 (m, 1H), 2.046 (m, 2H), 1.934 (s, 3H), 1.140 (s, 3H).

### Part C: Preparation of (4R)-menth-1-ene-3,8-diol [(6R)-6-(1-hydroxy-1-methylethyl)-3-methylcyclohex-2-en-1-ol]

A 100 mL Round bottom flask with magnetic stirrer bar was oven dried and cooled under stream of nitrogen. Lithium aluminum hydride (18 mmol, 0.68 g) and THF (20 ml) were added and resulting suspension is cooled to temperature 0° C.-5° C. The epoxide obtained from part B of example 2 (12 mmol, 2 g) was dissolved in 20 mL THF and added through addition funnel in 15 minutes. The mixture was stirred for 30 minutes to 0° C.-5° C. After completion of reaction as monitored by TLC, 5% aqueous sodium hydroxide was charged to quench the reaction mass. The resulting suspension is removed by filtration through the bed of hyflo. The obtained layer was extracted with methylene chloride (3×25 mL). Organic layer was washed with water and concentrated under reduced pressure. The product is further purified by column chromatography. Yield: 65%. IR: 3335, 2968, 1432 cm⁻¹. SOR 57.26 (c=0.5, EtOH), NMR δ 5.242 (s, 1H), 4.333 (m, 2H), 2.011 (m, 1H), 1.813 (m, 1H), 1.628 (s, 1H), 1.601 (s, 3H), 1.480 (m, 1H), 1.313 (s, 1H), 1.203 (s, 3H), 1.162 (t, 1H), 1.115 (s, 3H).

### Example 3: Process for Preparation of menthene-3,8-bis(diphenylacetyl ester)

Oven dried 250 mL round bottom flask with drying tube. Menth-1-ene-3,8-diol (64.6 mmol, 11 g) (from example 2, part C) was added, followed by pyridine (132 mL). The resulting solution was charged with dimethyl amino pyridine (12.9 mmol, 1.57 g) and stirred for thirty minutes at room temperature. Diphenyl acetyl chloride (209.3 mmol, 48.4 g) was added in lots within 15 minutes. The mixture was stirred at room temperature for sixty minutes. After completion of reaction, water (200 ml) was added, filtered off and obtained solid was re-dissolved in ethyl acetate (250 mL). Ethyl acetate layer was washed with aqueous 2% hydrochloric acid, aqueous sodium bicarbonate and water. It was then dried over sodium sulfate and concentrated under reduced pressure and further purified by stirring in 2-propanol. Yield: 64%. IR 1953, 1726, 1682, 1600, 1495, 1453, 1199, 1124 cm⁻¹; HPLC 99.56%; NMR: δ 7.264-7.143 (m, 20H), 5.3 (d, J=7.6 Hz, 1H), 5.158 (s, 1H), 4.851 (s, 1H), 4.775 (s, 1H), 2.132 (tt, 1H), 1.846-1.781 (m, 1H), 1.697-1.654 (m, 1H), 1.552 (s, 3H), 1.513-1.460 (m, 1H), 1.234 (s, 4H), 1.190 (s, 3H).

### Example 4: Process for Preparation of (−)-trans-Δ⁹-tetrahydrocannabinol

A 100 mL round bottom flask provided with magnetic stirrer bar and addition funnel was oven dried and cooled under stream of nitrogen. Menth-1-ene3,8-diol bis (diphenylacetyl) ester (example 3) (2.3 mmol, 1.3 g), Olivetol (2.3 mmol, 0.41 g) and Methylene chloride (39 mL) are added. The mixture was stirred and cooled at −10° C. BF₃.OEt₂ (2.3 mmol, 0.6 ml) is added by pressure equalizing funnel under nitrogen atmosphere. Progress is monitored by TLC. Reaction mass quenched with Aqueous 2% Sodium hydroxide. Methylene chloride layer was separated, dried over sodium sulfate. Obtained organic layer was concentrated to give light yellow syrup. Yield: 95%, HPLC: 92.8%. Crude (−)-trans-Δ⁹-tetrahydrocannabinol was purified by column chromatography, solvent used for elution from 1% DIPE-pet ether to 10% DIPE-pet ether. Yield: 65%, HPLC: 96.39% SOR: −149.36° (c=0.53 CHCl₃)

### Example 5: Process for Preparation of Menth-1-ene-3,8-diol from Menth-2-ene-1,8-diol [(1S,4R)-4-(1-hydroxy-1-methylethyl)-1-methylcyclohex-2-en-1-ol]

Part A: Preparation of (6S)-6-(1-hydroxy-1-methylethyl)-3-methylcyclohex-2-en-1-one. A 250 mL round bottom flask equipped with magnetic stir bar was charged with menth-2-ene-1,8-diol (29.4 mmol, 5 g), followed by 50 mL methylene chloride. To the resulting clear solution pyridinium chlorochromate (60.2 mmol, 13 g) was added at 15° C. in one lot. After completion of reaction, methylene chloride was removed under vacuum. Obtained slurry was stir with isopropyl ether. Organic layer was washed with water and concentrated under reduced pressure. Yield: 55%, IR: 3454, 2972, 1644, 1218, 1185 cm⁻¹. NMR: δ 5.772 (s, 1H), 2.3360-2.302 (m, 1H), 2.287-2.260 (m, 1H), 2.230 (d, 1H), 2.055-1.983 (m, 1H), 1.898 (s, 3H), 1.685-1.575 (m, 1H), 11.132 (s, 3H), 1.125 (s, 3H). Part B: Preparation of menth-1-ene-3,8-diol [(6R)-6-(1-hydroxy-1-methylethyl)-3-methylcyclohex-2-en-1-ol]. A 50 mL round bottom flask provided with magnetic stir bar was oven dried. Keto-alcohol (from part A, example 4) (8.92 mmol, 1.5 g) was added, followed by methanol (10 mL). The mixture was stirred at room temperature. Cerous chloride hepta hydrate (3.42 mmol, 1.25 g) was added and reaction mass was cooled to 0° C. Sodium borohydride (10.71 mmol, 0.4 g) was added, after completion of reaction, reaction mass was quenched with water and extracted with methylene chloride. The organic layer was concentrated under reduced pressure to give light yellow oil. Yield: 91%. SOR+136° (c=0.5, ethanol), GC: IR: 3357, 2970, 2829, 1293, 954 cm⁻¹. NMR: δ 5.502 (d, 1H, J=5.2 Hz), 4.349 (t, 1H, J=4 Hz), 3.690 (brs, 2H), 2.035-1.979 (m, 1H), 1.931-1.857 (m, 1H), 1.720-1.627 (m, 2H), 1.604 (s, 3H), 1.3 (s, 3H), 1.205-1.166 (m 1H), 1.070 (s, 3H).

### Example 6: Process for Preparation of menthene-3,8-bis(diphenyl acetyl) ester

Oven dried 250 mL round bottom flask with drying tube. Menthene-3, 8-diol (14.7 mmol, 2.5 g) (from example 5) was added, followed by pyridine (30 mL). Mixture was stirred at room temperature. Dimethyl amino pyridine (3 mmol, 0.36 g) was added to a clear solution. Diphenyl acetyl chloride (47.7 mmol, 11 g) was added in lots within 15 minutes. Stirring was continued at room temperature. After completion of reaction, water (50 ml) was added. It was filtered off and obtained solid was re-dissolved in ethyl acetate (50 mL). The Ethyl acetate layer was washed with Aq. Hydrochloric acid, aqueous sodium bicarbonate and water. It was dried over sodium sulfate and concentrated under reduced pressure and further purified by stirring in 2-propanol. Yield: 43%. IR 3068, 3029, 2967, 1724, 1492, 1453 cm¹; HPLC 98.44%; NMR: δ 7.238-7.136 (m, 20H), 5.6 (brs, 1H), 5.22 (brs, 1H), 4.85 (s, 1H), 4.77 (s, 1H), 2.18-2.10 (m, 1H), 1.94-1.85 (m, 1H), 1.71-1.82 (m, 1H), 1.57 (s, 3H), 1.39-1.52 (m, 1H), 1.25-1.35 (m, 1H), 1.155 (s, 3H), 1.063 (s, 3H).

### Example 7: Process for Preparation of (−)-trans-Δ⁹-tetrahydrocannabinol

A 100 mL round bottom flask provided with magnetic stirrer bar with addition funnel was oven dried and cooled under nitrogen. Menthene3,8-diol bis (Diphenyl ester) (example 6) (5.3 mmol, 3 g), Olivetol (5.3 mmol, 0.96 g) and Methylene chloride (90 mL) were added. The reaction mixture was stirred and cooled at −10° C. BF₃.OEt₂ (5.3 mmol, 1.4 ml) was added by pressure equalizing funnel under nitrogen atmosphere. The reaction progress was monitored by TLC. The reaction mass was then quenched with Aqueous 2% Sodium hydroxide. Methylene chloride layer was separated and dried over sodium sulfate. The obtained organic layer was concentrated. Yield: 97%, HPLC: 88.9%. The crude (−)-trans-Δ⁹-tetrahydrocannabinol was purified by column chromatography, solvent used for elution from 1% DIPE-pet ether to 10% DIPE-pet ether. Yield: 65%, HPLC: 95.64% SOR: −155.26° (c=0.53 CHCl₃).

### Example 8: Process for Preparation of (−)-trans-Δ⁹-tetrahydrocannabinol from menth-1-ene-3,8-diol, through its p-Ts carbamate

Part A: Preparation of menth-1-ene-3,8-bis[(4-methyl phenyl) sulfonylcarbamate]. A 100 mL round-bottom flask equipped with a magnetic stir bar and nitrogen inlet was charged with anhydrous methylene chloride (60 mL) and Menth-1-ene-3,8-diol (11.75 mmol, 2 g). To the above solution, p-Toluene sulfonyl isocyanate (29.3 mmol, 5.8 g) was added in 15 minutes controlling reaction temperature between 0° C.-5° C. Once menth-1-ene-3,8-diol was absent as monitored by TLC, reaction mass was quenched with aqueous ammonium chloride. The organic layer was dried over sodium sulfate.

Part B: Preparation of (−)-trans-Δ⁹-tetrahydrocannabinol.

A 250 mL round bottom flask provided with magnetic stirrer bar with addition funnel was oven dried and cooled under nitrogen flow of nitrogen. Organic layer from part-A and Olivetol (11.75 mmol, 2.1 g) was added to get a homogenous solution. The content of the flask was cooled to a temperature −5° C. to −10° C. BF₃.OEt₂ (11.75 mmol, 3.5 g, as on assay basis) was added in 5 minutes. The reaction mass was quenched with aqueous 2% sodium hydroxide. The organic layer was separated, dried over sodium sulfate and concentrated under reduced pressure to give oily syrup. Yield: 90%, HPLC: 87% (−)-trans-Δ⁹-tetrahydrocannabinol.

Part C: Purification: The crude compound was purified by column chromatography, solvent used for elution from 1% DIPE-pet ether to 10% DIPE-pet ether. Yield: 58%, HPLC: 96%.

Since many modification, variations and changes in detail can be made to the described embodiment/s of the invention, it is intended that all matters, in the foregoing description be interpreted as illustrative and not in a limiting sense. Thus, the scope of the invention should be determined by the appended claims and their legal equivalents.

1. A compound [C] having general formula as, 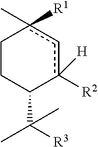
which is a derivative of olefins wherein
R¹: alkyl, aryl, alkyl aryl, heterocyclic, alkyl heterocyclic sulfonyl carbamate or H,
R²: alkyl, aryl, alkyl aryl, heterocyclic, alkyl heterocyclic sulfonyl carbamates or alkyl, aryl, Alkyl aryl, heterocyclic ester,
R³: alkyl, aryl, alkyl aryl, heterocyclic, alkyl heterocyclic sulfonyl carbamates or alkyl, aryl, Alkyl aryl, heterocyclic esters.

2. The compound as claimed in claim 1, wherein the olefin is one of menth-1-ene or menth-2-ene.

3. The compound as claimed in claim 1, wherein [C] is a compound of Formula IV, 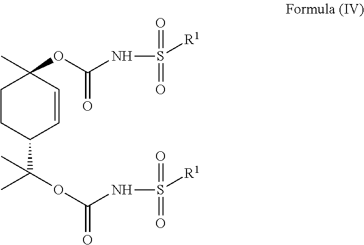
wherein R¹: n-Alkyl, branched alkyl, aryl, substituted aryl, alkyl aryl, substituted alkyl aryl (wherein substitution on alkyl or aryl or on both), heterocyclic derivatives, alkyl heterocyclic.

4. The compound as claimed in claim 3, wherein compound of Formula IV is compound of Formula XIII.

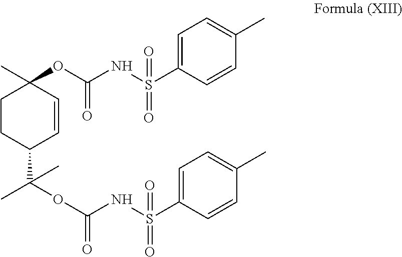 [SMILES: Cc1ccc(S(=O)(=O)NC(=O)OC(C)(C)[C@H]2C=C[C@](C)(OC(=O)NS(=O)(=O)c3ccc(C)cc3)CC2)cc1]

5. The compound as claimed in claim 1, wherein [C] is a compound of Formula XI. 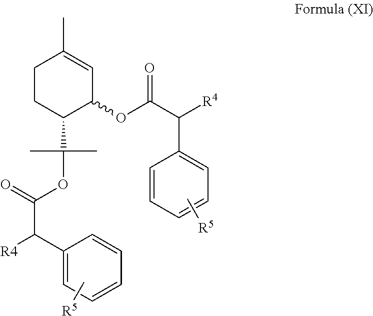
wherein R⁴ is at least one of H, aromatic, aliphatic, heteroaryl group, with or without substituents, the substituent is one or more of the group including Cl, Br, I, NO₂, SO₃H, R⁵ is at least one of H, Cl, Br, I, NO₂, SO₃H, alkyl, aryl or heterocyclic substituents connected via either carbon or heteroatom.

6. The compound as claimed in claim 5, wherein the compound of Formula XI is compound of Formula XIV

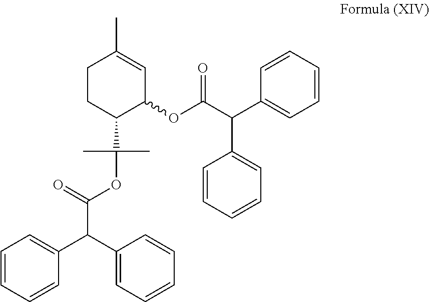 [SMILES: CC1=CC(OC(=O)C(c2ccccc2)c2ccccc2)[C@H](C(C)(C)OC(=O)C(c2ccccc2)c2ccccc2)CC1]

7. The compound as claimed in claim 1, wherein [C] is a compound of Formula VIII 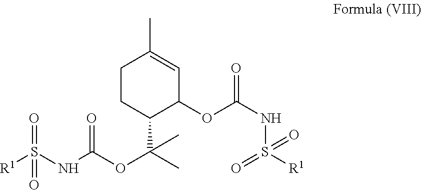
where in R¹ is aryl or heteroaryl.

8. The compound as claimed in claim 7, wherein the compound of Formula VIII is compound of Formula XV

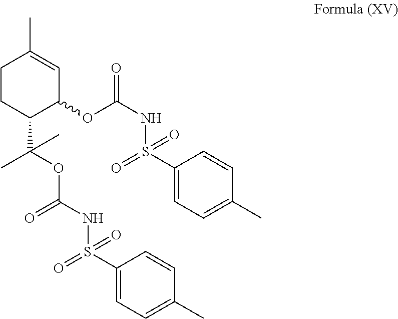 [SMILES: CC1=CC(OC(=O)NS(=O)(=O)c2ccc(C)cc2)[C@H](C(C)(C)OC(=O)NS(=O)(=O)c2ccc(C)cc2)CC1]

9. A process for the preparation of (−)-trans-Δ⁹-tetrahydrocannabinol with high purity and without sacrificing the atom economy comprising steps of:
preparing compound [C] of claim 1;
condensation of equimolar quantities of compound [C] with Olivetol (Formula V) carried out in organic solvent.

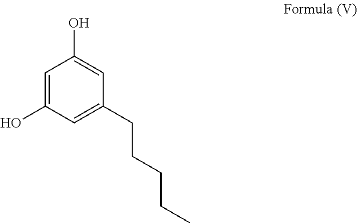

10. The process as claimed in claim 9, wherein the condensation of compound of [C] with Olivetol is carried out using any aromatic acid, mineral acids or Lewis acids or mixture thereof, preferably Lewis acids more preferably boron trifluoride etherate and preferably in the presence of dehydrating agents more preferably magnesium sulfate.

11. The process as claimed in claim 9, wherein compound used for condensation with olivetol is compound of Formula IV more particularly compound of formula XIII.

12. The process as claimed in claim 9, wherein compound used for condensation with olivetol is compound of Formula XI more particularly compound of formula XIV.

13. The process as claimed in claim 9, wherein compound used for condensation with olivetol is compound of Formula VIII more particularly compound of formula XV.

14. The process as claimed in claim 9, wherein during conversion to (−)-trans-Δ⁹-tetrahydrocannabinol, isomeric Δ⁸-tetrahydrocannabinol and cannabidiol as impurities were almost absent due to very short reaction time for cyclization.

15. A process for preparation of compound of Formula IV or VIII by condensation of menth-2-ene-1,8-diol or menth-1-ene-3,8-diol with alkyl/aryl/alkyl aryl/heteroaryl sulfonyl isocyanate, the reaction being carried out in organic solvents at temperature between −25° C. to +75° C.

16. The process as claimed in claim 15, wherein the alkyl/aryl/alkyl aryl/heteroaryl sulfonyl isocyanate is preferably phenyl sulfonyl isocyanates and more preferably p-toluene sulfonyl isocyanate.

17. The process as claimed in claim 15, wherein the reaction is carried out in organic solvent including tetrahydrofuran, dioxane, toluene, halogenated solvent or mixture thereof preferably halogenated solvents and more preferably methylene chloride.

18. The process as claimed in claim 15, wherein the reaction is carried out in the temperature between −25° C. to 75° C., preferably between −25° C. to 30° C. and more preferably between 0° C. to 20° C.

19. The process as claimed in claim 15, wherein the alkyl/aryl/alkyl aryl/heteroaryl sulfonyl isocyanate is p-toluene sulfonyl isocyanate and the compound formed is compound of Formula XIII or compound of Formula XV.

20. A process for preparation of the compound of Formula XI by acylation of menth-1-ene-3,8-diol in organic solvents using carboxylic acids or its respective acid chloride, in the presence of coupling agents and organic or weak inorganic bases.

21. The process as claimed in claim 20, wherein the carboxylic acid is aliphatic, aromatic, alkyl aryl, heterocyclic carboxylic acid preferably alkyl aryl carboxylic acid and more preferably benzyl, dibenzyl carboxylic acid or their respective acid chlorides.

22. The process as claimed in claim 20, wherein the coupling agents can be one of carbodiimides, Phosphonium based coupling agent (e.g. APO, PyAOP, BrOP, PyClop, FDPP, DEPBT, BDP), Uronium (e.g. BCC, TDBTU, TNU, TPTU, TSTU, HAPyu, TAPipU, CIP, HATU, TBTU), imminium based coupling agent (BOM, BDMP).

23. The process as claimed in claim 20, wherein organic solvents is one of Methylene chloride, ethylene chloride, toluene or organic bases preferably in pyridine (methyl pyridine and pyrrolidine) more preferably pyridine.

24. The process as claimed in claim 20, wherein the acid chloride is diphenyl acetyl chloride, solvent used is pyridine and the diester formed is compound of Formula XIV.

25. A process for preparing menth-1-ene-3,8-diol from menth-2-ene-1,8-diol. The method comprising steps of:
treating the menth-2-ene-1,8-diol with oxidizing agent in organic aprotic solvents to obtain ketone between −50° C. to +50° C.;
reducing the ketone obtained using hydride based reducing agent using Luche reduction condition in organic solvent.

26. The process as claimed in claim 25, wherein the oxidizing agent includes sodium chromate, potassium chromate, Pyridinium dichromate, pyridinium chlorochromate, potassium permanganate, manganese dioxide preferably pyridinium chlorochromate.

27. The process as claimed in claim 25, wherein hydride based reducing agents is one of NaBH₄, LiBH₄, KBH₄, NaBH₃CN, BH₃.THF or Aluminium hydrides as LiAlH₄, NaAlH₂(OC₂H₅OCH₃)₂ preferably sodium borohydride.

28. The process as claimed in claim 25, wherein Luche reduction condition means in presence of at least one of the compounds in a group comprising Cerium (III) chloride, Lanthanide (III) chloride, Scandium triflate preferably Cerium (III) chloride.
